(19) United States
(12) Patent Application Publication
(10) Pub. No.: US 2005/0234476 A1
(43) Pub. Date: Oct. 20, 2005

(54) Template grid needle/instrument guide and method for minimally invasive procedures

(76) Inventors: Willet F. Whitmore III, Longboat Key, FL (US); Roger F. Wilson, Sarasota, FL (US)

Correspondence Address: STEPTOE & JOHNSON LLP, 1330 CONNECTICUT AVENUE, N.W., WASHINGTON, DC, 20036, US

(21) Appl. No.: 11/027,495

(22) Filed: Dec. 29, 2004

Related U.S. Application Data
(60) Provisional application No. 60/532,554, filed on Dec. 29, 2003.
(60) Provisional application No. 60/631,086, filed on Nov. 24, 2004.

(51) Int. Cl. A61B 19/00

(52) U.S. Cl. 606/130

(57) ABSTRACT

A guide for elongate medical instruments includes a pair of cooperating members movable with respect to each other, each member having a receiving channel configured and dimensioned to receive one of said elongate medical instruments, wherein the receiving channels are parallel to one another. Each member may have a clamping surface coupled thereto for retaining the one of said elongate medical instruments in the receiving channel. Indicia may be provided for indicating separation distance between the receiving channels of the members. Methods of guiding elongate medical instruments into a patient also are disclosed.

### CROSS-REFERENCE TO RELATED APPLICATIONS

The benefits of Provisional Application No. 60/532,554 filed Dec. 29, 2003 and Provisional Application No. 60/631,086 filed Nov. 24, 2004 are claimed under 35 U.S.C. § 119(e), and the entire contents of these applications are expressly incorporated herein by reference thereto.

### FIELD OF THE INVENTION

The invention relates to a template grid needle/instrument guide and method for minimally invasive procedures.

### BACKGROUND OF THE INVENTION

Most so-called minimally invasive surgical procedures, such as biopsy, high dose rate (HDR) radiation, cryo-ablation, radio frequency (RF) ablation or drainage, are dependent on accurate initial needle or instrument placement into a mass lesion within the body. This critical initial placement typically is done using ultrasound, computed axial tomography (CT) or magnetic resonance imaging (MR) for guidance and confirmation of accurate positioning. For many procedures, placement of more than one needle or instrument is required for effective treatment. Depending on the treatment modality, it is frequently desirable to place additional needles or instruments in a spaced array surrounding the initial one, and in this manner to achieve a consistent spacing/positioning of needles or instruments within and/or around the lesion being treated. The ideal spacing of the instruments that deliver the therapy varies with the treatment modality and the tissue being treated and many details in this area are still under development. However, in every instance a predictable spacing is most desirable.

Currently almost all such procedures are done using a free-hand technique. With this approach, after placement of the initial needle or instrument, multiple additional passes with subsequent instruments and frequent or continuous real time imaging is used to achieve satisfactory spacing/positioning. In some cases, such as HDR brachytherapy of the prostate or cryotherapy of the prostate, a template grid that is initially rigidly fixed to an external device has been used to achieve accurate spacing and positioning of the treatment instruments. With this method the template is always held firm by an external device until all the instruments are in place.

There remains a need for a template grid needle/instrument guide and method for minimally invasive procedures that does not rely on externally placed devices to maintain a fixed position.

### SUMMARY OF THE INVENTION

The present invention relates to a guide for elongate medical instruments that includes a pair of cooperating members movable with respect to each other, each member having a receiving channel configured and dimensioned to receive one of said elongate medical instruments, wherein the receiving channels are parallel to one another. Each member may have a clamping surface coupled thereto for retaining the one of said elongate medical instruments in the receiving channel. Indicia may be provided for indicating separation distance between the receiving channels of the members.

The pair of cooperating members may be slidable with respect to each other, and each of the cooperating members may have a slot that receives a portion of the other cooperating member. In some embodiments, the pair of cooperating members includes (1) a first cooperating member with a slot formed therein and (2) a second cooperating member with a portion configured and dimensioned to be received in the slot. Also, the pair of cooperating members may be selectively lockable with respect to each other to resist relative movement and each clamping surface may be resiliently biased against the member. The indicia may include (1) a scale on a first of the cooperating members and (2) additional indicia on a second of the cooperating members alignable with the scale. The pair of cooperating members may be releasably secured to each other.

The present invention further relates to a guide for elongate medical instruments that includes a pair of cooperating members, each member including a portion slidably received in a slot in the other member, each member having a receiving channel configured and dimensioned to receive one of said elongate medical instruments and a clamping surface coupled to the member for retaining the one of said elongate medical instruments in the receiving channel. Indicia may be provided for indicating separation distance between portions of the members, and the receiving channels may have a fixed parallel orientation with respect to one another. The pair of cooperating members may be selectively lockable with respect to each other to resist relative movement, and each clamping surface may be resiliently biased against the member. The indicia may include (1) a scale on a first of the cooperating members and (2) additional indicia on a second of the cooperating members alignable with the scale.

In addition, the invention relates to a method of guiding elongate medical instruments into a patient using a guide comprising a pair of cooperating members that each define a region for receiving one of said instruments, the regions having a fixed parallel orientation with respect to one another, the method including: selectively adjusting the cooperating members so that the regions are disposed at a desired spacing with respect to each other; guiding a first of said instruments into the patient through a first of the regions of the guide; guiding a second of said instruments into the patient through a second of the regions of the guide so that the instruments are inserted into the patient parallel to one another. The method also may include releasably securing the first of said instruments to the guide.

In some embodiments, the method may further include: selectively readjusting the cooperating members so that the regions are disposed at another desired spacing with respect to each other; retaining one of said first and second of said instruments in one of the regions and guiding a third of said instruments into the patient through the other of the regions so that each of the instruments are inserted into the patient parallel to one another. The instruments all may be disposed in a straight line. In some embodiments, two of said instruments may be disposed equidistant from the other of said instruments. The second and third of said instruments, for example, may be disposed equidistant from the first of said instruments. A plurality of said instruments may be guided into the patient through the guide to be spaced equidistant from one of said instruments and disposed radially with respect to the one of said instruments. The instruments may be cryoprobes or needles.

Also, the present invention relates to a method of guiding elongate medical instruments into a patient using a guide that has a plurality of openings having a fixed parallel orientation with respect to one another, the method including: guiding a first of said instruments into the patient; placing the guide so that the first of said instruments extends through a first of the openings; guiding a second of said instruments into the patient through a second of the openings so that the instruments are inserted into the patient parallel to one another. In some embodiments, the guide comprises a pair of cooperating members and the method further comprises selectively adjusting the cooperating members so that the openings in the members are disposed at a desired spacing with respect to each other. The guide may be formed of a pair of cooperating members that each define at least one of said openings for receiving one of said instruments. The first of said openings may be a slot extending from a perimeter of the guide, or the first of said openings may be a central hole in the guide. The method may further include: coupling a plug to the first of said instruments; demountably coupling the plug in the first of the openings. In addition, the method may further include: guiding a third of said instruments into the patient through a third of the openings so that the instruments are inserted into the patient parallel to one another.

In accordance with one method of the present invention, an instrument such as a needle first may be inserted into a patient, and then a guide or grid subsequently may be coupled to the instrument for use in placing additional instruments in the patient. The guide or grid may facilitate placement of multiple instruments in a patient such that the instruments are disposed parallel to one another. In addition, arrays of instruments may be directed into a patient with such a grid or guide, thus for example permitting linear or radial positioning of instruments in a patient to be achieved.

The present invention further relates to a template grid and method for achieving accurate placement and spacing of needles/instruments that may be advantageous for minimally invasive therapies. Such a template grid may depend on an initially placed needle/instrument and then also may depend on subsequently placed needles or instruments for stability and positioning, rather than having its position held by an external device. In one exemplary embodiment, the template grid may be designed so that the initial needle/instrument may be placed or guided through it prior to initial placement within a lesion, or the template grid may be designed to attach to the initial needle/instrument after initial placement with respect to a lesion. In either case, the subsequent needles/instruments may be placed through the template grid to achieve accurate spacing and positioning in relation to the initially placed needle/instrument. The relative spacing and positioning of all needles/instruments that are placed after the initial one or two may vary as a function of the design of the template grid. The template grid may vary considerably in size, shape and dimension depending on the application. The template grid may or may not remain in place for the duration of a procedure or therapy.

### BRIEF DESCRIPTION OF THE DRAWINGS

Preferred features of the present invention are disclosed in the accompanying drawings, wherein:

FIG. 1A is a top perspective view of a first embodiment of a guide according to the present invention;

FIG. 1B is another top perspective view of the guide of FIG. 1A;

FIG. 1C is a bottom perspective view of the guide of FIG. 1A;

FIG. 1D is another bottom perspective view of the guide of FIG. 1A;

FIG. 1E is a front view of the guide of FIG. 1A;

FIG. 1F is a back view of the guide of FIG. 1A;

FIG. 1G is a top view of the guide of FIG. 1A;

FIG. 1H is a bottom view of the guide of FIG. 1A;

FIG. 1I is a left view of the guide of FIG. 1A;

FIG. 1J is a right view of the guide of FIG. 1A;

FIG. 2 is a side perspective view of a second embodiment of a guide according to the present invention;

FIG. 3A is a top view of a third embodiment of a guide according to the present invention in an unassembled state;

FIG. 3B is a top view of the guide of FIG. 3A in an assembled state;

FIG. 4A is a top view of a fourth embodiment of a guide according to the present invention;

FIG. 4B is a side perspective view of the guide of FIG. 4A with a split plug for use therewith;

FIG. 5A is a top perspective view of a fifth embodiment of a guide according to the present invention; and

FIG. 5B is a bottom perspective view of the guide of FIG. 5A.

### DETAILED DESCRIPTION OF THE PREFERRED EMBODIMENTS

Terms such as “top,” “bottom,” and “side” as used herein are provided as a non-limiting examples of the orientation of features.

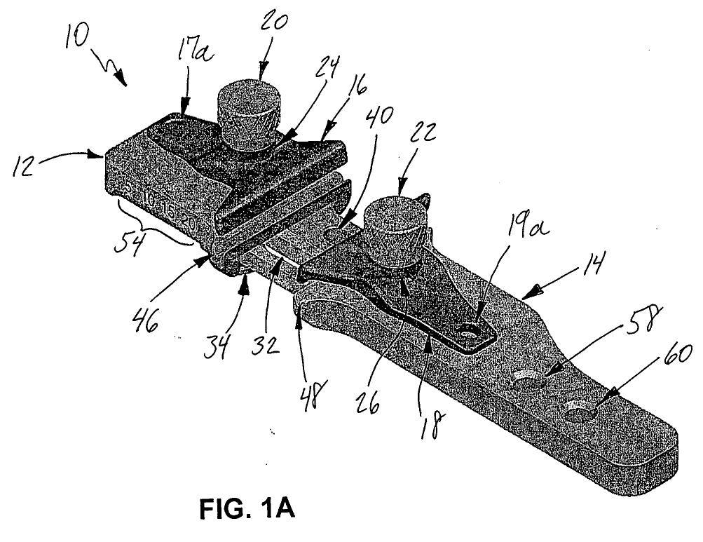

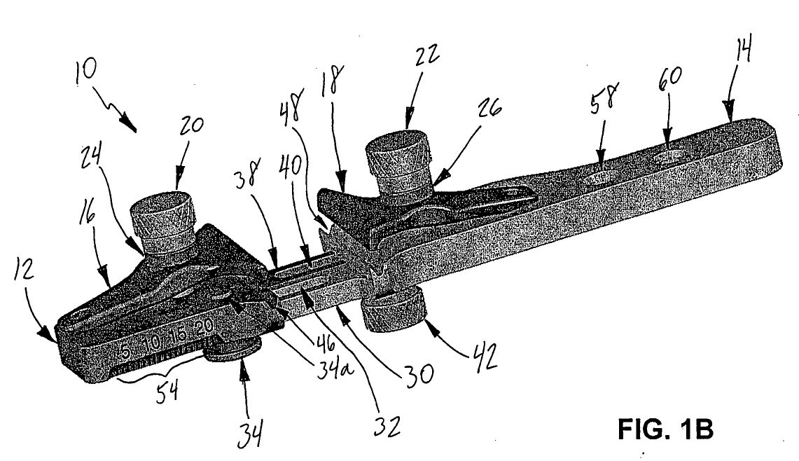

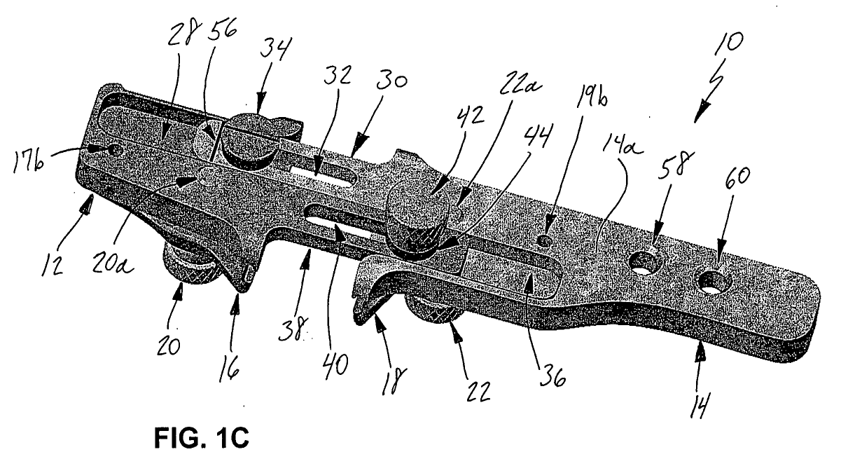

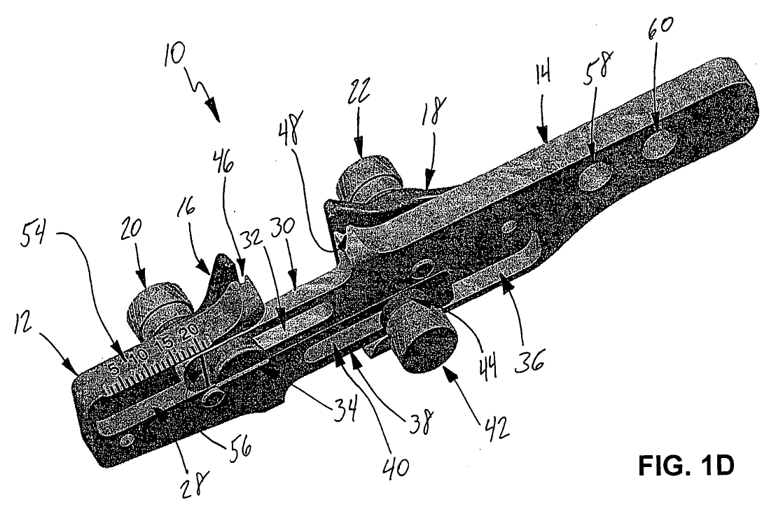

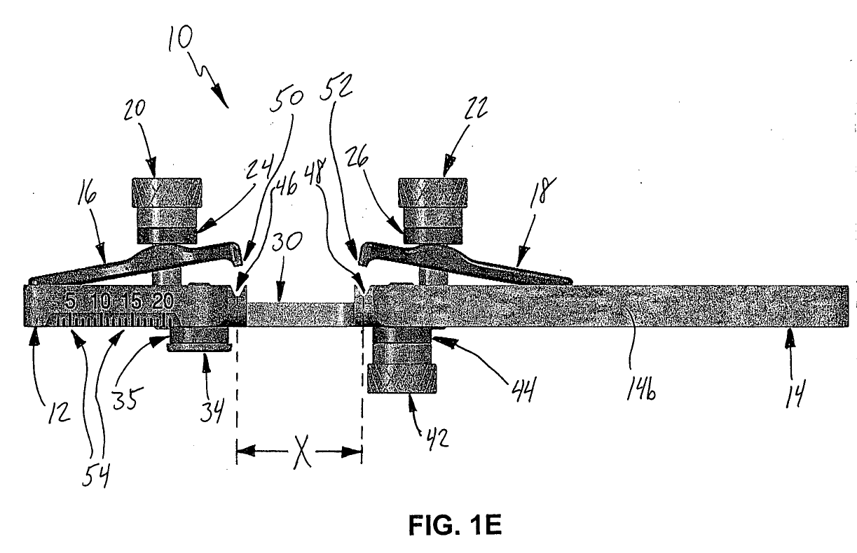

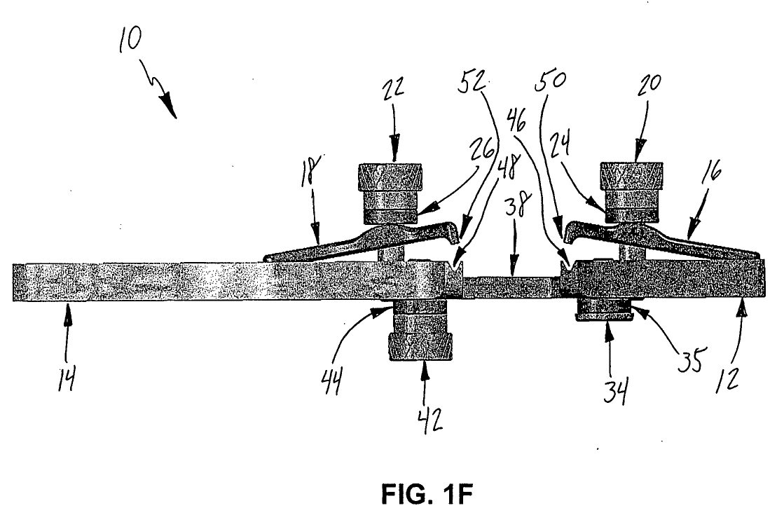

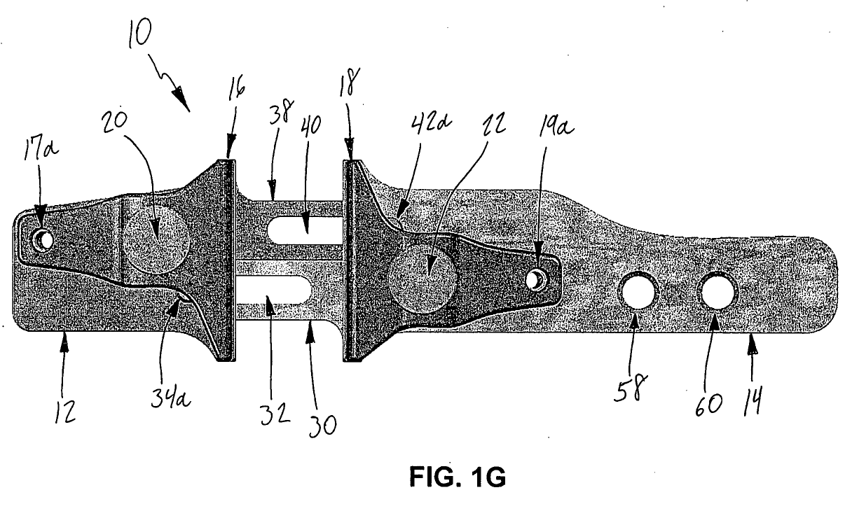

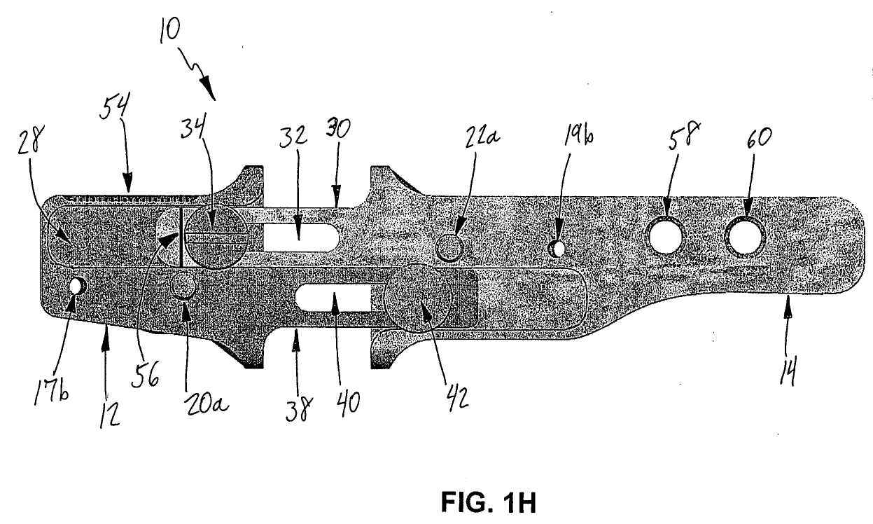

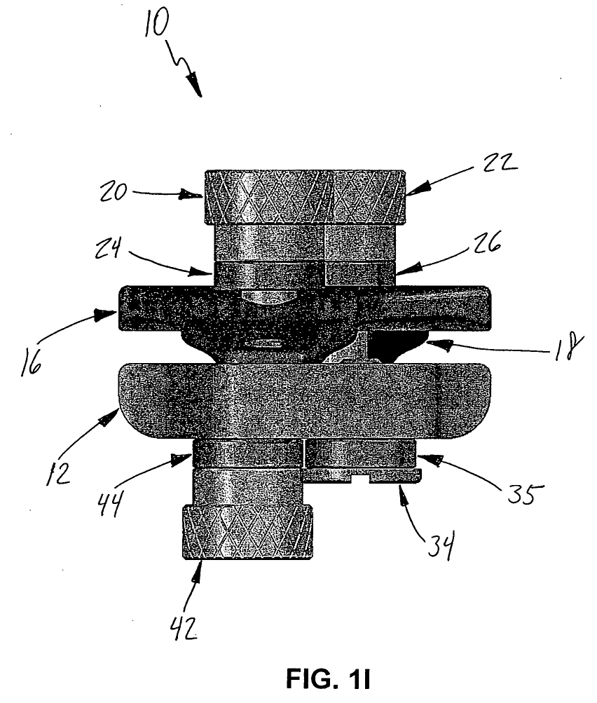

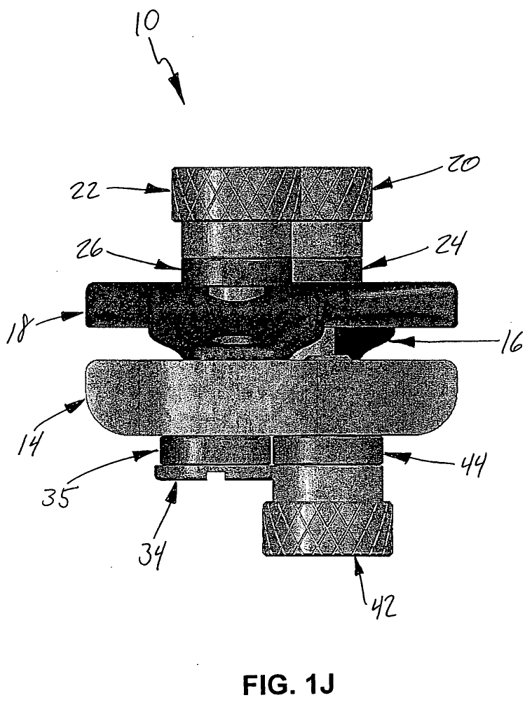

Referring initially to FIGS. 1A-1J, a preferred exemplary embodiment of a guide 10 according to the present invention is shown. Guide 10 includes cooperating slide guides 12, 14 with clamps 16, 18, respectively, coupled thereto. Thumb screws 20, 22 and corresponding washers 24, 26 adjustably couple clamps 16, 18 to guides 12, 14. In particular, thumb screws 20, 22 are threadably received in threaded holes 20a, 22a in guides 12, 14, respectively. In addition, optionally holes 17a, 19a may be provided in clamps 16, 18 and aligned with corresponding threaded holes 17b, 19b in slide guides 12, 14. Such additional holes provide a further means for securing clamps 16, 18 to slide guides 12, 14, as for example with threaded screws (not shown). Slide guide 12 includes a slot 28 in which an elongate portion 30 of slide guide 14 is slidably received. A slot 32 in elongate portion 30 of slide guide 14 receives a set screw 34 therein which is threadably received in a hole 34a in slide guide 12. Set screw 34 is provided with a corresponding washer 35. Thus, slide guide 14 is demountably coupled to slide guide 12 with set screw 34. In addition, slide guide 14 includes a slot 36 in which an elongate portion 38 of slide guide 12 is slidably received. A slot 40 in elongate portion 38 of slide guide 12 receives a thumb screw 42 therein which is mounted on a washer 44 and threadably received in a hole 42a in slide guide 14. Thus, slide guide 12 is demountably coupled to slide guide 14 with thumb screw 42.

To assist in adjusting thumb screws 20, 22, 42, surface texturing and preferably knurling is provided on the heads thereof. Also, set screw 34 may be provided for example in a Phillips-head configuration to facilitate adjustment of the tightness of coupling slide guide 14 to slide guide 12 such as by using a screw driver. In addition, thumb screws 20, 22 for example may be 10-32 aluminum screws, while thumb screw 42 may be an 8-32 aluminum screw.

Clamps 16, 18 preferably are configured, dimensioned and formed of a suitable material to provide resilient biasing against corresponding slide guides 12, 14. In particular, when a surface proximate an end of each clamp 16, 18 abuts a slide guide 12, 14, and when thumb screws 20, 22 are installed to bear against clamps 16, 18, respectively, the clamps are biased away from slide guides 12, 14.

Slots 28, 36 preferably are substantially parallel to one another, and are sized to accommodate elongate portions 30, 38, respectively. Moreover, elongate portions 30, 32 preferably are configured and dimensioned to be guided within slots 28, 36, respectively, and preferably are guided substantially parallel to one another.

Slide guides 12, 14 have elongate grooves 46, 48, respectively, disposed proximate free ends thereof, and clamps 16, 18 have elongate protruding edges 50, 52 that oppose grooves 46, 48 respectively. In the preferred exemplary embodiment, grooves 46, 48 are V-shaped, but in alternate embodiments the grooves may have other geometries such as arcuate shapes. Preferably, grooves 46, 48 are sized to accommodate linear instruments of a variety of sizes. For example, grooves 46, 48 may be sized to receive elongate instruments between 18 gauge and 2.5 mm in diameter. When an elongate instrument abuts a groove 46, 48, the instruments may be releasably secured in the groove by moving clamps 16, 18 so that a protruding edge 50, 52 abuts the instrument. The position of the clamping surface created by protruding edge 50, 52 is adjustable due to thumb screws 20, 22, respectively, which may be operated to bear down on clamps 16, 18 to decrease the separation distance between a groove 46, 48 and its corresponding clamp 16, 18 with edge 50, 52. Preferably, when slide guides 12, 14 are coupled to each other as indicated, the respective grooves 46, 48 therein are aligned parallel to each other. Thus, linear instruments placed in grooves 46, 48 may be disposed parallel to one another.

By tightening a thumb screw 20, 22 while an elongate instrument is disposed in a corresponding groove 46, 48, the instrument may be tightly retained in a given position in the groove. Alternatively, if less pressure is applied by the thumb screw the instrument may be secured within the groove but loosely retained so that movement of the instrument substantially parallel to the groove is freely permitted. This user selectable feature for example permits flexibility in needle/instrument placement during minimally invasive procedures.

Advantageously, slide guides 12, 14 are slidably associated with each other in a predetermined relationship such that a known, predictable spacing may be achieved between grooves 46, 48 and thus elongate instruments disposed therein. In particular, a separation distance X between grooves 46, 48 may be set by a user. Slide guide 12 includes indicia 54 in the form of a scale, preferably disposed on a side thereof. In one preferred embodiment, slide guide 12 includes a scale for indicating separation distances X between about 3 mm and about 21 mm. As can be seen for example in FIGS. 1C, 1D, 1H, slide guide 14 also may be provided with indicia 56, preferably in the form of a line or arrow oriented generally perpendicular to slot 32 and alignable with the scale of indicia 54. Alternatively, indicia 56 may be a dot or other feature, and optionally may be scored on slide guide 14. As shown for example in FIG. 1C, indicia 56 is provided on a bottom surface of 14a of slide guide 14 so as to be visual to a user when guide 10 is assembled. However, the location of indicia 54, 56 as disclosed is preferred but non-limiting. In an alternate embodiment, indicia instead may be provided on one or more of slide guides 12, 14, and may even be provided on a side surface such as surface 14b as shown in FIG. 1E. The indicia is provided for the purpose of indicating a known, predetermined spacing between grooves 46, 48. When set screw 34 and/or thumb screw 42 is loosened to permit sliding of elongate portion 30 of slide guide 14 within slot 28 of slide guide 12, a user may select a desired separation distance X, for example from about 3 mm to about 21 mm. Set screw 34 and/or thumb screw 42 subsequently may be tightened to releasable fix the position of elongate portion 30 in slot 28 and thus releasably fix the separation distance X. Advantageously, by placing a first elongate instrument such as a needle in a first groove 46, 48, a second elongate instrument may be placed a known or predetermined separation distance X away from the first elongate instrument. And preferably the first and second elongate instruments may be placed parallel to one another. The first elongate instrument serves effectively as the guide for the placement of the second elongate instrument. Instrument placement during minimally invasive procedures thus may proceed in a registered/indexed fashion.

In addition, this procedure may be repeated for example so that after the second elongate instrument is placed, the clamping action on one of the first and second elongate instruments is released and the other elongate instrument is then used as the guide for placing one or more additional elongate instruments. In particular, the separation distance X may be changed so that another elongate instrument may be placed at yet another separation distance. Alternatively, the same separation distance may be maintained while additional elongate instruments are placed in a radial fashion with respect to one of the first and second elongate instruments.

Guide 10, for example, may be hand-held, attached to a guidance device, or held by a mechanical positioning arm to establish the positioning thereof with respect to a patient. Guide 10 permits elongate instruments such as needles to be captured and released as desired to create radial or rectilinear placement arrays.

A pair of holes 58, 60 also may be provided in guide 10 for mounting, as may be desired, to a guidance tool such as a stereotactic guidance device for initial needle placement. In one non-limiting exemplary embodiment, both holes 58, 60 are provided in slide guide 14.

In the preferred exemplary embodiment, components of guide 10 are formed of aluminum, although some or all components also may be formed of stainless steel. In alternate embodiments, some or all of the components may be formed of polymer.

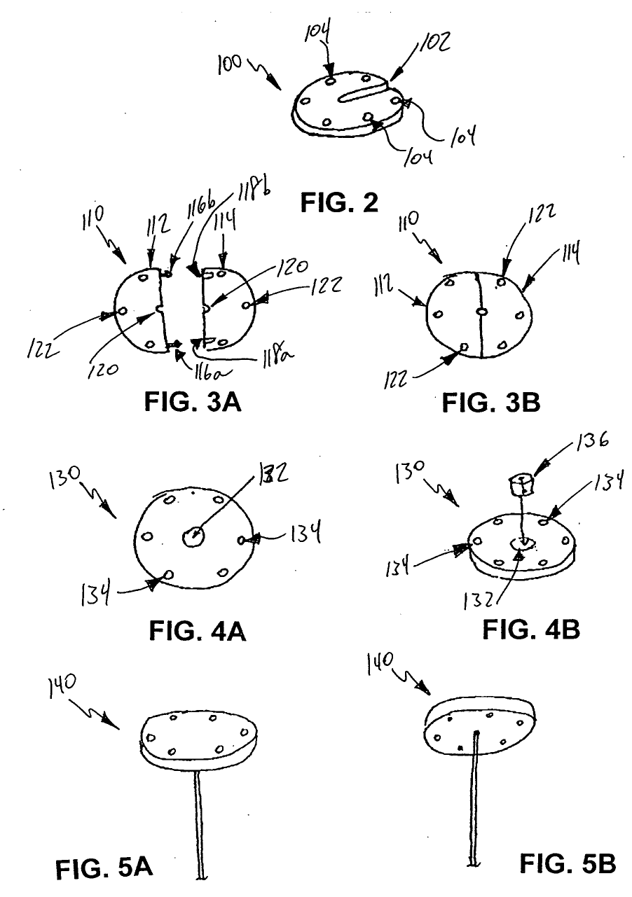

Turning to FIG. 2, another exemplary embodiment of a template grid 100 according to the present invention is shown. In particular, guide 10 is generally circular with a through slot 102 extending from a perimeter thereof to proximate the center thereof. Holes 104 are provided in a radial fashion about the perimeter. The holes and slot are configured and dimensioned for guiding elongate instruments therein, preferably parallel to one another.

In another exemplary embodiment of a template grid 110 according to the present invention, shown in FIGS. 3A-3B, a generally circular guide is provided by two interlocking halves 112, 114. A pair of pegs 116a, 116b are received in corresponding grooves 118a, 118b, respectively. A central hole 120 is formed by the two opposing halves. Additional radially disposed holes 122 also are provided. The holes are configured and dimensioned for guiding elongate instruments therein, preferably parallel to one another.

In yet another embodiment of a template grid 130 according to the present invention as shown in FIGS. 4A-4B, a circular guide is provided with a central hole 132 and radially disposed holes 134. A split plug 136 also is provided with a slot for engaging a centrally disposed instrument and for mounting in central hole 132. The holes and slot are configured and dimensioned for guiding elongate instruments therein, preferably parallel to one another.

In addition, another exemplary embodiment of a template grid 140 is shown in FIGS. 5A-5B. A central needle is coupled to one side of a circular guide, and through-holes for receiving additional instruments are radially disposed with respect thereto. The holes are configured and dimensioned for guiding elongate instruments therein, preferably parallel to one another.

In an exemplary application of the template grids and methods of the present invention, a four centimeter renal tumor is treated using for example a template grid of FIGS. 2-5. A tumor of such size is too large to be treated with a single cryoprobe and may require as many as six or more probes for complete treatment. To achieve uniform freezing, it is desirable to have the probes accurately spaced and running parallel to each other and to place temperature sensors at strategic locations around the tumor. A guide, possibly with a particular thickness, and with appropriate hole spacing, is attached to an initial probe after placement thus allowing for rapid and accurate subsequent placement of multiple parallel probes and surrounding sensors.

Prior art template grids for needle/instrument placement are used following initial positioning and stabilization by an external device. However, in some embodiments of the present invention, the template grid is positioned and stabilized by the needle(s)/instrument(s) placed into the patient. The mechanical means to accomplish this type of template grid and method of use, as shown for example in FIGS. 2-5, include but are not limited to: (1) a slot that may engage an initially placed needle/instrument, (2) a multipart template grid that comes together to engage an initially placed needle/instrument, (3) a template grid configured to fit over or around an initially placed needle/instrument, (4) a template grid designed to have the needle/instrument passed through it prior to initial placement, and (5) a template grid with a needle/instrument attached to it.

All template grids and guides used in invasive applications must be sterile or else not come in direct or indirect contact with the patient. Since these grids and guides by design will come in direct or indirect contact via the needle/instrument or by resting on the surface of the body or an organ in open surgery, they must be sterilizable and preferably be available to the user pre-sterilized and disposable.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. For example, the clamping surfaces of clamps 16, 18 may be spring-loaded or otherwise resiliently biased away from slide guides 12, 14. Also, slide guides 12, 14 instead may be coupled to each other with only one elongate portion received in one slot. Each slide guide 12, 14 may be provided with more than one groove for receiving elongate instruments such as needles; for example, three grooves may be provided proximate a free end of the slide guide. At least one of the slide guides may be received in an enclosed slot extending within the other slide guide, rather than in an open slot as discussed with respect to guide 10. Further, a ratchet arrangement may be provided such that movement of slide guides 12, 14 with respect to each other is indexed. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

1. A guide for elongate medical instruments comprising:
a pair of cooperating members movable with respect to each other, each member having a receiving channel configured and dimensioned to receive one of said elongate medical instruments;
wherein the receiving channels are parallel to one another.

2. The guide of claim 1, wherein the pair of cooperating members are slidable with respect to each other.

3. The guide of claim 1, wherein each of the cooperating members comprises a slot that receives a portion of the other cooperating member.

4. The guide of claim 1, wherein the pair of cooperating members comprises (1) a first cooperating member with a slot formed therein and (2) a second cooperating member with a portion configured and dimensioned to be received in the slot.

5. The guide of claim 1, wherein the pair of cooperating members are selectively lockable with respect to each other to resist relative movement.

6. The guide of claim 1, wherein each member further comprises a clamping surface coupled thereto for retaining the one of said elongate medical instruments in the receiving channel.

7. The guide of claim 1, wherein each clamping surface is resiliently biased against the member.

8. The guide of claim 1, further comprising indicia for indicating separation distance between the receiving channels of the members.

9. The guide of claim 1, wherein the indicia comprises (1) a scale on a first of the cooperating members and (2) additional indicia on a second of the cooperating members alignable with the scale.

10. The guide of claim 1, wherein the pair of cooperating members are releasably secured to each other.

11. A guide for elongate medical instruments comprising:
a pair of cooperating members, each member including a portion slidably received in a slot in the other member, each member having a receiving channel configured and dimensioned to receive one of said elongate medical instruments and a clamping surface coupled to the member for retaining the one of said elongate medical instruments in the receiving channel;
indicia for indicating separation distance between portions of the members;
wherein the receiving channels have a fixed parallel orientation with respect to one another.

12. The guide of claim 11, wherein the pair of cooperating members are selectively lockable with respect to each other to resist relative movement.

13. The guide of claim 11, wherein each clamping surface is resiliently biased against the member.

14. The guide of claim 11, wherein the indicia comprises (1) a scale on a first of the cooperating members and (2) additional indicia on a second of the cooperating members alignable with the scale.

15. A method of guiding elongate medical instruments into a patient using a guide comprising a pair of cooperating members that each define a region for receiving one of said instruments, the regions having a fixed parallel orientation with respect to one another, the method comprising:
selectively adjusting the cooperating members so that the regions are disposed at a desired spacing with respect to each other;
guiding a first of said instruments into the patient through a first of the regions of the guide;
guiding a second of said instruments into the patient through a second of the regions of the guide so that the instruments are inserted into the patient parallel to one another.

16. The method of claim 15, further comprising:
releasably securing the first of said instruments to the guide.

17. The method of claim 15, further comprising:
selectively readjusting the cooperating members so that the regions are disposed at another desired spacing with respect to each other;
retaining one of said first and second of said instruments in one of the regions and guiding a third of said instruments into the patient through the other of the regions so that each of the instruments are inserted into the patient parallel to one another.

18. The method of claim 17, wherein the instruments are all disposed in a straight line.

19. The method of claim 17, wherein two of said instruments are disposed equidistant from the other of said instruments.

20. The method of claim 17, wherein the second and third of said instruments are disposed equidistant from the first of said instruments.

21. The method of claim 17, wherein a plurality of said instruments are guided into the patient through the guide to be spaced equidistant from one of said instruments and disposed radially with respect to the one of said instruments.

22. The method of claim 17, wherein the instruments are cryoprobes.

23. The method of claim 17, wherein the instruments are needles.

24. A method of guiding elongate medical instruments into a patient using a guide comprising a plurality of openings having a fixed parallel orientation with respect to one another, the method comprising:
guiding a first of said instruments into the patient;
placing the guide so that the first of said instruments extends through a first of the openings;
guiding a second of said instruments into the patient through a second of the openings so that the instruments are inserted into the patient parallel to one another.

25. The method of claim 24, wherein the guide comprises a pair of cooperating members and the method further comprises selectively adjusting the cooperating members so that the openings in the members are disposed at a desired spacing with respect to each other.

26. The method of claim 24, wherein the guide comprises a pair of cooperating members that each define at least one of said openings for receiving one of said instruments.

27. The method of claim 24, wherein the first of said openings comprises a slot extending from a perimeter of the guide.

28. The method of claim 24, wherein the first of said openings comprises a central hole in the guide.

29. The method of claim 24, further comprising:
coupling a plug to the first of said instruments;
demountably coupling the plug in the first of the openings.

30. The method of claim 24, further comprising:
guiding a third of said instruments into the patient through a third of the openings so that the instruments are inserted into the patient parallel to one another.

31. The method of claim 30, wherein the instruments are all disposed in a straight line.

32. The method of claim 30, wherein two of said instruments are disposed equidistant from the other of said instruments.

33. The method of claim 30, wherein the second and third of said instruments are disposed equidistant from the first of said instruments.

34. The method of claim 30, wherein a plurality of said instruments are guided into the patient through the guide to be spaced equidistant from one of said instruments and disposed radially with respect to the one of said instruments.

35. The method of claim 30, wherein the instruments are cryoprobes.

36. The method of claim 30, wherein the instruments are needles.
